(19) United States
(12) Patent Application Publication
(10) Pub. No.: US 2008/0195185 A1
(43) Pub. Date: Aug. 14, 2008

(54) Thermal therapy device for post-surgery recovery

(76) Inventors: Benjamin J. Krempel, San Francisco, CA (US); Annie B. Tucker, San Francisco, CA (US); Eric C. Selvik, Menlo Park, CA (US); Michael Thomas Derse, San Francisco, CA (US)

Correspondence Address: LUMEN PATENT FIRM, INC., 2345 YALE STREET, SECOND FLOOR, PALO ALTO, CA, 94306, US

(21) Appl. No.: 11/998,866

(22) Filed: Nov. 30, 2007

Related U.S. Application Data
(63) Continuation-in-part of application No. 11/784,033, filed on Apr. 4, 2007, now abandoned.
(60) Provisional application No. 60/791,303, filed on Apr. 12, 2006.

(51) Int. Cl. A61F 7/00

(52) U.S. Cl. 607/108

(57) ABSTRACT

A device for providing thermal therapy for post-surgery or post-injury recovery of the breast region is provided. The thermal therapy device includes a bladder for allowing temperature-controlled fluid to flow throughout the bladder and a shell for holding the bladder. The bladder can include holes to increase its conformability to irregular three-dimensional shapes. One or more splints on the shell help to stabilize the breasts and a layer provides insulation to the bladder. The device is designed to be separable in the front, thereby allowing it to be easily worn and removed.

### CROSS-REFERENCE TO RELATED APPLICATIONS

This application is a continuation-in-part of U.S. Non-Provisional patent application Ser. No. 11/784,033 filed Apr. 4, 2007. U.S. Non-Provisional patent application Ser. No. 11/784,033 filed Apr. 4, 2007 is cross-referenced to and claims benefit of U.S. Provisional Patent Application 60/791,303 filed Apr. 12, 2006. U.S. Non-Provisional patent application Ser. No. 11/784,033 filed Apr. 4, 2007 is cross-referenced to and claims benefit of U.S. Non-Provisional patent application Ser. No. 11/064,546 filed Feb. 23, 2005. U.S. Non-Provisional patent application Ser. No. 11/064,546 filed Feb. 23, 2005 is cross-referenced to and claims benefit of U.S. Provisional Patent Application 60/546,903 filed Feb. 23, 2004. All of which are hereby incorporated by reference.

### FIELD OF THE INVENTION

The invention relates generally to temperature-controllable devices. More particularly, the present invention relates to therapeutic temperature-controllable devices for post-surgery recovery.

### BACKGROUND

A person who has undergone surgery or experienced injury generally requires treatment to facilitate healing of the affected region of the body. Typically, the application of cold to the affected region is desired to decrease swelling and bruising in the region. Cold treatment has been found to be particularly useful after cosmetic surgeries, such as breast augmentations. Traditional devices to serve this purpose are generally based on using ice-filled or gel-filled packs. The packs first must be frozen and then applied to the affected region to provide cold relief.

The frozen nature of the packs creates at least three major disadvantages to such devices. First, a pack is initially at or below a freezing temperature, which can potentially cause frostbite or, at the very least, discomfort to the user of the pack. Due to the freezing temperature, the pack must be intermittently removed from the body, thereby reducing the therapeutic effect. The second disadvantage of a frozen pack is that the pack melts and increases in temperature while it is being used. As the temperature is constantly changing, the degree of effectiveness of the device changes. In other words, the device cannot provide consistent thermal therapy to the user. Lastly, hot therapy is sometimes desired for post-surgery or post-injury treatment. Clearly, a frozen ice or gel pack cannot provide heat therapy to affected regions.

Traditional devices can also be ineffective when applied to irregularly shaped body parts, such as breasts. For instance, gel packs in their frozen state cannot conform to the affected body part. Furthermore, the application of the packs to the irregular body part requires tape or another fastener means. Using complicated fastening methods could be difficult for an injured person. Alternatively, an assistant may be required to help a user apply and fasten the packs onto the affected areas. This requirement could cause embarrassment, especially when privacy of the affected regions is desired, such as for the breast regions.

In addition to cold therapy, post-surgery treatment generally requires stabilization of and/or application of pressure to the affected regions. Movement of the affected parts can disrupt the normal healing process. A disrupted healing process can lead to scars and undesirable visible markings. This result is particularly troubling when the purpose is primarily for aesthetics, as it is in cosmetic surgery.

The present invention addresses the difficulties of providing post-surgery treatment to irregularly shaped regions of a body, especially the breasts, and advances the art with a wearable device for providing thermal therapy.

### SUMMARY OF THE INVENTION

The present invention provides a thermal therapy device for post-surgery or injury recovery of the breast region of a human subject. The device provides cooling or heating therapy to breast regions that have recently undergone a surgical procedure, such as breast augmentation surgery. The thermal therapy device provides consistent temperature treatment and stabilization to the affected regions and the device is conformable to the breasts.

The thermal therapy device is composed of a breast pad bladder and a breast pad shell. The bladder includes a region to cover the right breast, a region to cover the left breast, a conduit region connecting the right and left regions, and a passageway for fluid to flow throughout all of the regions. The temperature of the fluid is controllable and can stay approximately constant. The bladder is conformable to irregular shapes, particularly the breasts. To increase conformability, the bladder can have holes near the center of the left and right regions. Movable flaps with fluid passageways connected to the temperature-controlled fluid can also be included with the bladder.

The breast pad shell holds the bladder onto a subject and enables the device to be wearable and comfortable. The shell includes a layer adhered to the bladder and one or more splints conformable to the breasts. The layer can provide insulation to the fluid. A splint stabilizes a breast during recovery. Additional layers and straps can be attached to the shell to provide comfort.

The device is designed to be portable and easily worn and discarded. In particular, the conduit region is shaped to fit around the back of the torso of the subject to allow the left and right regions to be separable in the front. A fastener, such as a zipper, is used to fasten the two regions. This design enables a recovering subject to wear and remove the device with little or no assistance.

### BRIEF DESCRIPTION OF THE FIGURES

The present invention together with its objectives and advantages will be understood by reading the following description in conjunction with the drawings, in which:

FIG. 1 shows an example of a breast pad bladder according to the present invention.

FIG. 2 shows an example of a breast pad shell according to the present invention.

FIG. 3 shows a schematic lateral cross section of an example of a thermal therapy device according to the present invention.

### DETAILED DESCRIPTION OF THE INVENTION

Post-surgery treatment or treatment to injured areas of the body helps to facilitate the healing process. Treatment generally requires applying cold or heat to the damaged areas and stabilizing the areas. For regions of the body with complex shapes, such as the breasts, the application of treatment could be difficult. Below is a detailed description of a device for providing thermal therapy to the breasts of a human subject.

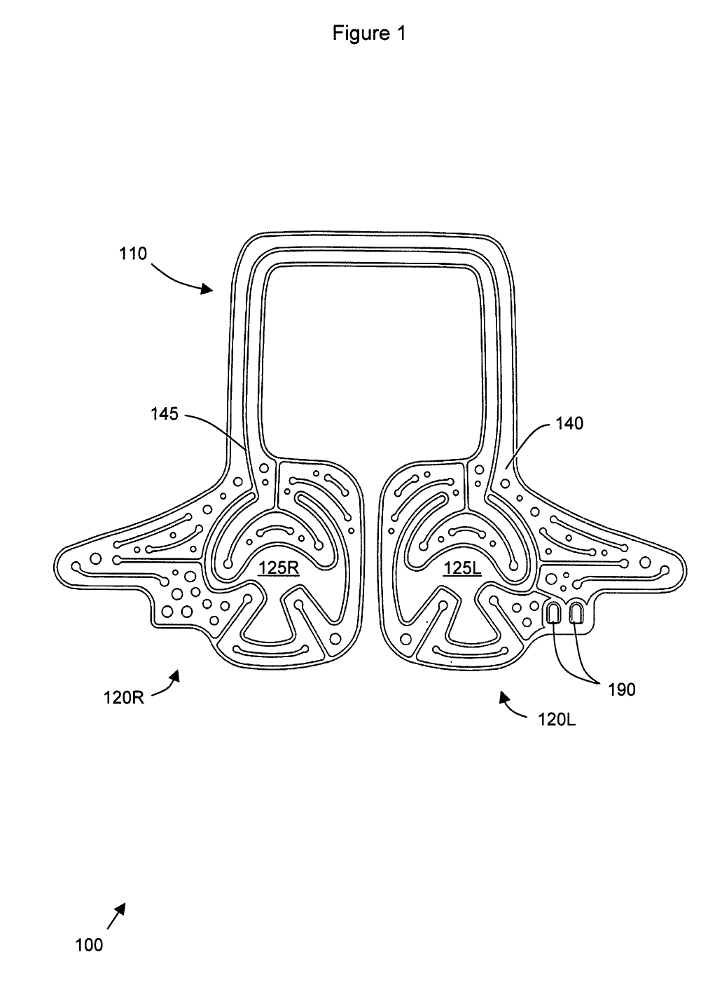

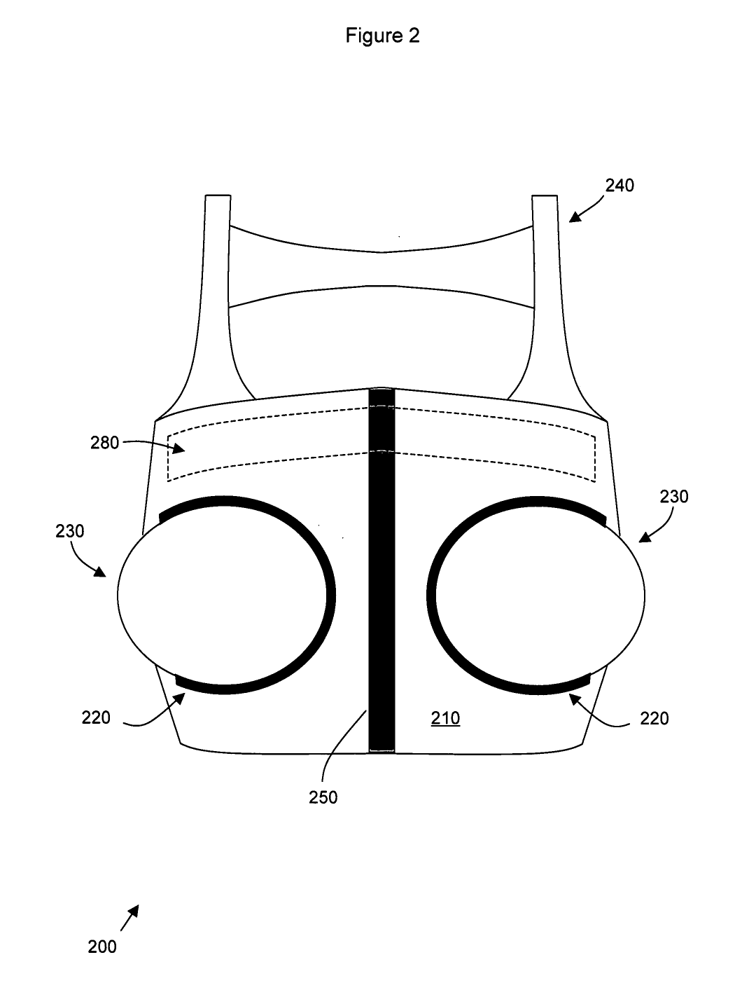

A portable therapeutic temperature-controllable device for post-surgery treatment or injury treatment allows the device to be used at the convenience of the injured subject, including inside the subject's home and outside of hospitals and clinics. A comfortable and portable thermal therapy device for the breasts is composed of a breast pad bladder, shown in FIG. 1, and a breast pad shell, shown in FIG. 2.

FIG. 1 shows an example of a breast pad bladder 100 for delivering therapy to a subject. The breast pad bladder 100 is placed on or near a subject to absorb heat from the subject or deliver heat to the subject. The breast pad bladder 100 covers a large area of the breasts and includes a right region 120R, a left region 120L, and a conduit region 110. Fluid passes through the conduit region 110 as it is transported from the left region 120L to the right region 120R or vice versa. The fluid passageways 140 with passageway walls 145 provide channels for the fluid to be transported through. The fluid passageways 140 are made from vinyl, polyvinyl chloride, rubber, urethane, polyurethane, polyethylene, neoprene, silicone, a hydrogel, a hydrocolloid, or a mixture thereof. Further details of the fluid passageways can be found in U.S. Non-Provisional patent application Ser. No. 11/784,033 filed Apr. 4, 2007, which is hereby incorporated by reference.

Cooling fluid is transported through the fluid passageways 140 to provide thermal therapy to a subject in contact with the regions of the breast pad bladder 100. The fluid flow may be constant or pulsatile. The fluid passageways may also have valves to control the fluid flow inside of the passageways. The fluid can optionally contain active agents.

In a preferred embodiment, the conduit region 110 is sized and positioned to be worn around the back of the torso of the subject instead of directly in between the right 120R and left 120L regions. This location of the conduit region 110 allows the right 120R and left 120L regions to be separated, thereby the thermal therapy device, generally, and the breast pad bladder, specifically, can be easily worn and taken off by the subject. The breast pad bladder 100 also has inlet/outlet features 190 to receive/transmit fluid into and out of the bladder. In a preferred embodiment, the inlet/outlet features 190 are conveniently placed on one side of the subject's body.

FIG. 1 also shows right 125R and left 125L cutout portions located near the center of the right 120R and left 120L regions, respectively. The cutout portions serve two purposes. First, the portions enable the two-dimensional breast pad bladder 100 to be conformable to the three-dimensional shape of the breasts. Second, the cutout portions prevent the nipples and their surrounding areas from receiving cold or hot therapy. Without the cutout portions, the sensitivity of the nipples may irritate the subject or hinder blood flow to the nipples.

In an embodiment, flaps are included with the breast pad bladder 100. The flaps can be any size or shape and are preferably constructed with fluid passageways similar to the breast pad bladder 100 in FIG. 1. The fluid passageways of the bladder 140 can be fluidically connected with the fluid passageway of the flaps. The flaps are positionable in multiple configurations, one or more of which allows delivery of thermal therapy to the desired area. In some circumstances, thermal therapy may be desired for the sensitive areas, including the cutout nipple areas of the breasts. User-positionable flaps allow the subject to provide or remove therapeutic relief to the sensitive areas at the subject's behest.

FIG. 2 shows an example of a breast pad shell 200 for keeping the breast pad bladder 100 in place on the subject to provide thermal therapy. The breast pad bladder 100 is adjacent to the subject and the shell 200 is worn over the bladder 100. In a preferred embodiment, the bladder 100 and shell 200 are adhered to each other. Shoulder straps 240, which may be adjustable, allow the subject to comfortably wear the thermal therapy device. In a preferred embodiment, a front fastener, such as a zipper 250, is placed in the front of the breast pad shell 200 to keep the shell together. In addition to or in replacement of a zipper, the front fastener may also be a hook and loop fastener, a snap, a river, a button, lace, or any other fastener means. The front fastening technique allows a subject to put the pad on alone or with little assistance.

The breast pad shell includes a layer 210 that covers the breast pad bladder. Preferably, the layer 210 is an insulating layer for efficiently retaining heat or cold inside of the thermal therapy device. The insulating layer helps keep cold or heat on the subject's body instead of wastefully allowing the heat or cold to escape. The breast pad shell 200 may also include pads 230 made of a stretchable material, such as Lycra. The pads 230 fit over the breasts of the subject and can accommodate different-sized breasts due to their stretchiness.

It is important to note that the breast pad shell also includes one or more splints 220 conformable to the breasts. The splints 220 are used to comfortably hold the breasts or implants. Besides providing comfort, splints 220 also serve in stabilizing the breasts or implants while the subject undergoes healing from an injury or post-surgery. Decreasing the motion of the breasts due to movement of the user helps the healing process. In a preferred embodiment, the splints 220 are essentially C-shaped and positioned to stabilize around the perimeter of the breasts. The C-shape enables both the upper and lower regions of the breast or implant to be supported and pressure to be applied to the regions. The splint 220 can also be adjustable and reformable. The splint 220 is made from a thermoplastic material, a metal, copper, aluminum, stainless steel, brass, tin, a ferrous metal, a plastic, an alloy, a wax, rubber, or a mixture thereof. For additional support and comfort, one or more compression straps 280, attachable to the breast pad shell, can also be provided.

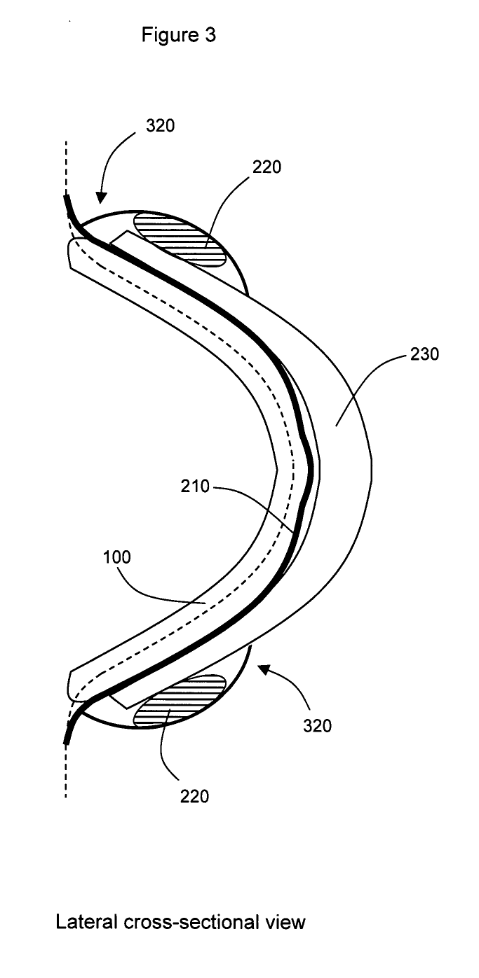

FIG. 3 shows a schematic lateral cross-section of the breast region of a thermal therapy device. The breast pad bladder 100 is in contact with and applying thermal therapy to a breast of the subject. The dashed lines in FIG. 3 outline the breast. A layer 210 is adhered to the bladder 100 for holding the bladder 100 and providing insulation. A stretchable pad 230 is located on the layer 210 and provides comfort and fit for the breast. A splint 220 for supporting the breast of the subject is located on the upper and lower edges of the pad 230. In the cross-sectional view shown in FIG. 3, a single C-shaped splint 220 appears as two separate segments. The thermal therapy device can optionally include splint covers 320. In another embodiment not shown in the figures, the splints stay fixed in pockets of the insulating layer.

As one of ordinary skill in the art will appreciate, various changes, substitutions, and alterations could be made or otherwise implemented without departing from the principles of the present invention, e.g. fluids of any temperature can be used in the device and the device can include additional functional or decorative layers or flaps. Accordingly, the scope of the invention should be determined by the following claims and their legal equivalents.

What is claimed is:

1. A thermal therapy device, comprising:
a breast pad bladder formed to at least partially cover a right breast and a left breast of a subject, wherein said breast pad bladder comprises:
a right region for at least partially covering the right breast;
a left region for at least partially covering the left breast;
a conduit region; and
a fluid passageway, wherein said fluid passageway extends from said right region to said left region via said conduit region and the fluid is permitted to flow therethrough; and
a breast pad shell for holding said breast pad bladder on the subject, wherein said breast pad shell comprises:
a layer adhered to said breast pad bladder; and
a reformable splint.

2. The device as set forth in clam 1, wherein said conduit region is shaped to fit around the back of a torso of the subject.

3. The device as set forth in claim 1, wherein said device can be connected and disconnected in between the right breast and the left breast by a fastener, wherein said fastener is a zipper, a hook and loop fastener, a snap, a rivet, a button, lace or a combination thereof.

4. The device as set forth in claim 1, further comprising at least one user positionable flap, wherein said flap comprises an active fluid passageway, wherein said flap can be opened and closed.

5. The device as set forth in claim 4, wherein said flap can be positioned to cover and uncover at least a portion of a breast of the subject, including the nipple portion.

6. The device as set forth in claim 1, wherein said breast pad bladder is conformable to the right breast and the left breast by having a right hole located near the center of the right region and a left hole located near the center of the left region.

7. The device as set forth in claim 1, wherein said splint is made at least in part from a thermoplastic material, a metal, copper, aluminum, stainless steel, brass, tin, a ferrous metal, a plastic, an alloy, a wax, rubber or a mixture thereof.

8. The device as set forth in claim 1, wherein said fluid passageway region is made at least in part from vinyl, polyvinyl chloride, rubber, urethane, polyurethane, polyethylene, neoprene, silicone, a hydrogel, a hydrocolloid or a mixture thereof.

9. The device as set forth in claim 1, wherein said reformable splint is positioned near the perimeter of either the right or left region of the breast pad bladder and wherein said splint extends from the bottom of the same region to the top of the same region along the perimeter of the same region.
